(19) United States
(12) Patent Application Publication
(10) Pub. No.: US 2016/0187328 A1
(43) Pub. Date: Jun. 30, 2016

(54) DEVICES, SYSTEMS, AND METHODS FOR MULTIPLEX ALLERGEN TESTING

(71) Applicant: Himanshu C. Mody, Springville, UT (US)

(72) Inventor: Himanshu C. Mody, Springville, UT (US)

(21) Appl. No.: 14/942,890

(22) Filed: Nov. 16, 2015

Related U.S. Application Data
(60) Provisional application No. 62/080,151, filed on Nov. 14, 2014.

(51) Int. Cl. G01N 33/543

(52) U.S. Cl. CPC G01N 33/54306

(57) ABSTRACT

Multiplex devices, systems, and associated methods for assaying a biological sample for an allergen-binding molecule are disclosed and described.

### PRIORITY DATA

This application claims the benefit of U.S. Provisional Patent Application Ser. No. 62/080,151, filed on Nov. 14, 2014, which is incorporated herein by reference in its entirety.

### BACKGROUND

An allergic reaction is a hypersensitive response of the immune system to an allergen, which results in the hyperactivation of mast cells and basophils by immunoglobulin E (IgE) antibodies. Symptoms associated with the resulting inflammatory response can be wildly varied, and can range from mildly irritating to life threatening. Allergens can be environmental, insect- or animal-related, dietary, drug-related, etc.

Current allergen testing in the United States, for example, is generally an in vivo testing procedure using an array of allergen exposures on the skin of a subject. This procedure can be inconclusive, particularly for many mild allergic reactions, partly due to the subjective nature of the determination and the potential difficulties in ascertaining a positive skin reaction resulting from a mild reaction or irritation. Such in vivo testing can also be uncomfortable because a positive result is the generation of an allergic reaction in the subject. Other technologies commonly used include in vitro testing of a single or very limited number of allergens per assay.

### BRIEF DESCRIPTION OF THE DRAWINGS

For a fuller understanding of the nature and advantage of the present disclosure, reference is being made to the following detailed description of various embodiments and in connection with the accompanying drawings, in which:

FIG. 1 shows a graphical representation of a reaction well according to one aspect of the present disclosure.

FIG. 2 shows a graphical representation of a multiwell plate according to one aspect of the present disclosure.

### DETAILED DESCRIPTION

Although the following detailed description contains many specifics for the purpose of illustration, a person of ordinary skill in the art will appreciate that many variations and alterations to the following details can be made and are considered to be included herein.

Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term in this specification, like “comprising” or “including,” it is understood that direct support should be afforded also to “consisting essentially of” language as well as “consisting of” language as if stated explicitly and vice versa.

“The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein.

As used herein, “enhanced,” “improved,” “performance-enhanced,” “upgraded,” and the like, when used in connection with the description of a device or process, refers to a characteristic of the device or process that provides measurably better form or function as compared to previously known devices or processes. This applies both to the form and function of individual components in a device or process, as well as to such devices or processes as a whole.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. However, it is to be understood that even when the term “about” is used in the present specification in connection with a specific numerical value, that support for the exact numerical value recited apart from the “about” terminology is also provided.

Further, it is to be understood that the listing of components, species, or the like in a group is done for the sake of convenience and that such groups should be interpreted not only in their entirety, but also as though each individual member of the group has been articulated separately and individually without the other members of the group unless the context dictates otherwise. This is true of groups contained both in the specification and claims of this application. Additionally, no individual member of a group should be construed as a de facto equivalent of any other member of the same group solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### EXAMPLE EMBODIMENTS

An initial overview of technology embodiments is provided below and specific technology embodiments are then described in further detail. This initial summary is intended to aid readers in understanding the technology more quickly, but is not intended to identify key or essential technological features, nor is it intended to limit the scope of the claimed subject matter.

Allergic responses can range from mild irritations to life threatening circumstances. It can be highly beneficial to determine an allergy profile for a given subject, whether the subject is a human or a non-human mammal capable of allergic reaction. For example, a great number of children have allergic reaction to peanuts and peanut products, which can be elicited by the mere contact with a person having recently eaten such products. In cases where this allergy is unknown, a life threatening reaction can occur for which there has been no preparation. Furthermore, humans that experience even mild allergies may find that these reactions reduce their effectiveness at work, their concentration, and even their enjoyment of life. Avoiding allergens that trigger such reactions would improve their productivity and outlook. As another example, a valuable racehorse may exhibit variations in performance due to an unknown allergy to a food product ingredient. Removal of the allergen from the horse's food can thus improve the performance and value of the horse.

Given the potential for the general improvement relating to performance, happiness, and wellbeing, including potentially life-saving benefits, it is surprising that the state of the art of allergy testing has not developed significant technologies to rapidly and effectively screen a subject for the many hundreds of known allergens. In vivo testing using an array of allergen exposures on the skin of a subject, for example, is still widely used in the United States. This procedure can be inconclusive, particularly for many mild allergic reactions, partly due to the subjective nature of the determination and the potential difficulties in ascertaining a positive skin reaction resulting from a mild reaction. Such in vivo testing can also be uncomfortable because a positive result is the generation of an allergic reaction in the subject. Other technologies commonly used include in vitro testing of a single or very limited number of allergens per assay. There has thus been a very long felt need for rapid allergen testing of a biological sample from a subject that has not been met by the industry.

The present disclosure provides devices, systems, and methods for rapidly assaying for allergens (i.e. analytes) in a biological sample that are indicative of a potential allergy in a subject from which the sample was taken. This assaying technology allows for the testing of a vast array of allergens and/or allergen panels simultaneously in a true multiplexing assay format. The ability to simultaneously test very large numbers of allergens in vitro can be used to identify one or more potential allergy in a subject without the need to generate an allergic response. Because the disclosed methodology has the potential of quickly assaying such large numbers of allergens, an accurate allergic profile can readily be generated for a given subject. Additionally, such testing can elucidate allergic response distinctions between related allergens to provide a more highly detailed allergic profile. Furthermore, multiplex testing facilitates the simultaneous screening of these large numbers of allergens or allergen panels that would be costly and time consuming using single allergen tests, and would likely be impossible using either in vivo methodologies or single allergen testing procedures.

In general, multiplex testing can be defined as a device, system, or method that can simultaneously test for multiple analytes in the same reaction solution, and in many cases using the same or similar testing reagents. Such multiplex testing can therefore be used to assay a large number of analytes while minimizing reagents and other components of the reaction process. It is noted, however, that the present scope is not limited by reaction solution and/or reagent composition or commonality. For example, in some aspects multiple different analytes are assayed simultaneously in the same solution and using the same reagents. In other aspects, multiple different analytes are assayed simultaneously in the same solution and using different reagents. In such cases, multiple reagents can be added to the same solution such that different analytes may be assayed for simultaneously, but with different reagents. In yet other aspects, multiple different analytes can be assayed in a common solution enclosure, but at different points in time. For example, a reaction solution can be added to a solution enclosure (e.g., a well in a multi-well plate), and a first reagent or reagents can be used to assay for one or more analytes in a sample. Subsequent reagent or reagents can then be added to the reaction solution to assay for one or more additional analytes in the sample. It is further contemplated that the testing of analytes at different points in time can be accomplished by removing and replacing the reaction solution between analyte assays.

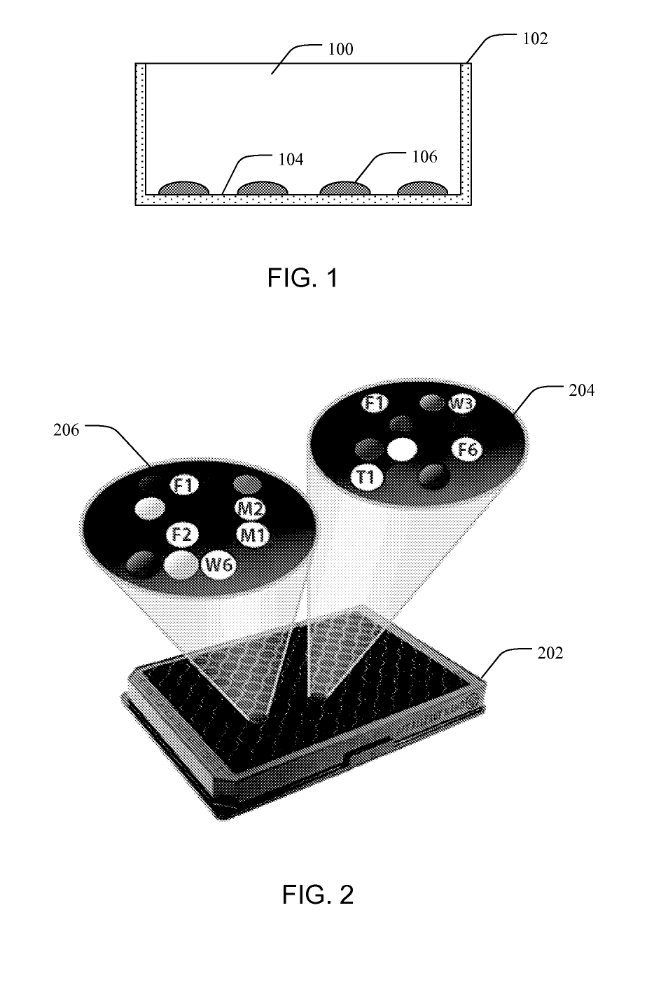

In one aspect, a device for assaying a biological sample for an allergen-binding molecule is provided. Such a device can include a multi-well plate having a multiple reaction wells with a binding surface at a bottom portion of each well. A plurality of discrete spots of allergen material are coupled to the binding surface of at least one of the reaction wells, where at least two discrete spots in at least one reaction well include different allergen material. One non-limiting example of a reaction well is shown in FIG. 1, and includes a housing 102 that defines the sides of the reaction well 100, and a binding surface 104. In one aspect, the binding surface can be an extension of the housing. In another aspect, the binding surface can be distinct from the housing, and in some cases can be removable from the housing. In those cases where the housing is a multiwell plate or other housing having multiple reaction wells, for example, the binding surface can be a single structure that is removed from across the entire multiwell plate, or the binding surface can be comprised of multiple sections or structures that can be independently removed from the multiwell plate.

Furthermore, discrete spots of allergens 106 are shown coupled to the binding surface 104 of the reaction well 100. In referring to such spots, the term “allergen” includes an allergen as understood in the art, a portion such allergen, a synthetic molecule or compound capable of functioning as the allergen in the assay, a tether molecule that binds to the allergen as a step in the manufacturing or in the protocol for using the assay, and the like. The spots of allergens can be disposed on the binding surface in a variety patterns, pattern configurations, and/or allergen combinations depending on the design of the multiwell plate, the nature of a given assay, the preferences of the assay designer, and the like, all of which are considered to be within the present scope. For example, in one aspect a given well can include multiple spots of the same allergen. In another aspect, a well can include multiple spots of the same allergen at different concentrations, different purities, different forms of the same allergen, and/or the like. In yet another aspect, a well can include multiple spots of different allergens. Different allergens can include unrelated allergens, as well as related allergens, or a combination thereof. For example, it can be beneficial to include related allergens in a well that represent a panel of allergens. A panel can be any desired grouping of allergens. In some cases, an allergen panel can be a grouping of allergens from a related category, such as grass allergens, nut allergens, animal allergens, milk product allergens, dust allergens, and the like. In other cases, a panel can be a grouping of allergens associated with a given location, such as home allergens, work allergens, geographical location allergens, and the like. Other panels can include childhood allergens, food allergens, seasonal allergens, and the like. Furthermore, in some aspects one or more wells in a multiwell plate can include a positive control spot, while in other aspects, each well can contain such a control spot.

The arrangements of allergen spots across the wells in a multiwell plate can also vary depending on the design of the multiwell plate, the nature of a given assay, the preferences of the assay designer, and the like, all of which are considered to be within the present scope. For example, in one aspect multiple wells in a multiwell plate can contain the same allergens, allergen combinations, and/or allergen panels. In some aspects, all wells being used to assay allergens can contain the same allergens, allergen combinations, and/or allergen panels. In one assay design, each well can contain the same allergen panel, and each sample processed in each well can be from a different individual. In this manner, multiple individuals can be simultaneously tested for the same allergen panel. In other aspects, two or more wells in a multiwell plate can contain different allergens, allergen combinations, and/or allergen panels, and in yet other aspects all wells used to assay allergens can contain different allergens, allergen combinations, and/or allergen panels. While a multiwell plate having different allergens in each well can be used to test a sample from a single individual across the entire plate, samples from different individuals can also be tested.

It is additionally contemplated that the spots of allergens can be arranged such that relevant allergy data can be obtained from the pattern of positive and/or negative spots across a single reaction well, multiple reaction wells, or the entire multiwell plate. Allergens can thus be positioned across the binding surface such that the relative spacing between various allergens is intended to provide informational indicators that may not be apparent merely from the positive and negative reactions of those allergens alone. For example, plant allergens can be patterned across the binding surface according to taxonomical groupings, but can also have one or more spatial patterns that represent other allergy-relevant groupings. Non-limiting examples of such groupings for plant allergens may include indoor vs. outdoor plants, regional locations for the plants that produce the allergens, seasonal diversities, groupings related to common foods the allergens may be used in, and the like. Any arrangements of allergens that provide allergy-relevant data from the spatial pattern allergens is considered to be within the present scope.

FIG. 2 shows one example of a multiwell plate 202, and expanded views of two of the reaction wells, 204 and 206. Various spots of allergens are shown in the expanded views. Thus a single reaction solution can be used in each well to assay numerous allergens simultaneously.

As has been described, allergens are coupled to the binding surface of a well in a multiwell plate. The formation of the allergen spots can be accomplished by any known method of applying a material to be tested to a substrate, provided the material remains secure when exposed to the assay solution, during the assay, and during any subsequent analysis. Non-limiting examples of such techniques can include jet printing, pipetting, direct transfer, and the like. In some cases, the allergen material can be directly applied to the binding surface, while in other cases a linking or tether molecule can be applied to the binding surface that has an affinity for the allergen material or to a linking molecule coupled to the allergen material. Additionally, other non-allergen material can be included in the spot of allergen material, whether mixed with the allergen prior to application or subsequently added following spotting. Such non-allergen material can be ingredients used to facilitate handling and/or deposition, adhesion, stability, improve shelf life, and the like.

Any form of assay or assay technique capable of detecting the presence of an antibody or other allergy-related biomarker can be used with the presently disclosed multiwell plates, and each are considered to be within the present scope. Non-limiting examples of such assays include various immunoassays, enzyme, enzyme-linked, sandwich, fluorescent assays, and the like. Specific examples can include enzyme immunoassays (EIA) and enzyme-linked immunoassays (ELISA), including variations such as ELISA sandwich assays. Other specific aspects can include fluorescent and radio labeled immunoassays.

What is claimed is:

1. A device for assaying a biological sample for an allergen-binding molecule, comprising:
a multiwell plate having a plurality of reaction wells;
a binding surface at a bottom portion of each of the plurality of reaction wells; and
a plurality of discrete spots of allergen material coupled to the binding surface of at least one of the plurality of reaction wells, wherein at least two discrete spots in at least one reaction well include different allergen material.

2. The device of claim 1, wherein each of the plurality of reaction wells includes discrete spots of different allergens.

3. The device of claim 1, wherein the plurality of discrete spots is arranged as a plurality of allergen panels across the plurality of reaction wells.

4. The device of claim 3, wherein the spots of allergen material in each of the plurality of reaction wells is an allergen panel.

5. The device of claim 1, wherein the binding surface is removable from the multi-well plate.

6. The device of claim 5, wherein the binding surface further comprises multiple independently removable sections.
